(19) United States
(12) Patent Application Publication
(10) Pub. No.: US 2010/0152534 A1
(43) Pub. Date: Jun. 17, 2010

(54) CAPSULE ENDOSCOPY SYSTEM, MEDICAL SYSTEM, AND OPERATION METHOD OF MEDICAL SYSTEM

(76) Inventors: You Jin KIM, Daejeon (KR); Hyung Soo LEE, Daejeon (KR); Jae Young KIM, Daejeon (KR); Kwang Roh PARK, Daejeon (KR); Hong Soon NAM, Daejeon (KR)

Correspondence Address: LADAS & PARRY LLP, 224 SOUTH MICHIGAN AVENUE, SUITE 1600, CHICAGO, IL, 60604, US

(21) Appl. No.: 12/507,164

(22) Filed: Jul. 22, 2009

(30) Foreign Application Priority Data
Dec. 16, 2008 (KR) 10-2008-0127809

(51) Int. Cl. A61B 1/045; G08B 21/00

(52) U.S. Cl. 600/109; 340/870.16

(57) ABSTRACT

Provided are a capsule endoscopy system, a medical system, and an operation method of the medical system. A control device outside a human body and two or more implantable medical devices (e.g. capsule endoscopes) form a network with each other, and the two or more implantable medical devices divisionally perform investigation, identification, and treatment while cooperating with each other, by controlling the control device outside the human body.

### CROSS-REFERENCE TO RELATED APPLICATION

This application claims priority to and the benefit of Korean Patent Application No. 10-2008-0127809 filed in the Korean Intellectual Property Office on Dec. 16, 2008, the entire contents of which are incorporated herein by reference.

### BACKGROUND OF THE INVENTION

(a) Field of the Invention

The present invention relates to a capsule endoscopy system, a medical system, and an operation method of the medical system. In detail, the present invention relates to a capsule endoscopy system that performs examination, identification, and treatment of the inside of a human body, a medical system, and an operation system of the medical system.

(b) Description of the Related Art

The inside of a human body is formed in a layered structure of bones, internal organs, skin, etc., and as the human moves, different internal organs continuously have organic interaction. Further, due to various changes of living things, the inside of a human body is made of very complicate structures where chemical, physical, and electrical actions are generated.

Therefore, it is required for medical devices that are used inside a human body to ensure not only safety, but diagnostic accuracy.

In generally, medical devices that are used inside a human body include an implantable medical device (hereafter referred to as “IMD”) that performs functions such as data collection and image pick-up in a human body, and a controller that transmits/receives data to/from the IMD at the outside of the human body. The IMD performs a function of transmitting data to the controller using wireless communication, and as an IMD, a capsule endoscope that picks up images of the inside of a human body, such as the stomach and the small intestine, and transmits data to a controller using wireless communication, has been known. Further, the controller is a device that is wearable on a body or placed in a wireless body area network (hereafter referred to as “WBAN”) within 2 to 3 m from the human body, and performs functions of controlling the IMD or receiving data from the IMD.

For example, in a capsule endoscope, a capsule-type device is inserted inside a human body through the patient's esophagus, and it picks up images of the inside of the human body, particularly the small intestine, and then transmits data to a controller using wireless communication. The images picked up by the capsule type endoscope are stored by the controller, and medical personnel determine whether a disease exists on the basis of the stored data.

Meanwhile, in the related art, an IMD typically operates independently and diagnoses the state of specific parts, and a technology of diagnosis and treatment using two or more independent IMDs which cooperate with each other has not been proposed. However, safety and accuracy are required for diagnosis and treatment of the inside of a human body, such that it may be more advantageous for the diagnosis and treatment of a disease to perform diagnosis and treatment using two or more IMDs that cooperate with each other, as compared with using one IMD. Accordingly, it is required to develop a technology for diagnosis and treatment of a state of the inside of a human body using two or more IMDs that cooperate with each other.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a capsule endoscopy system, a medical system, and an operation method of the medical system having advantages of effectively providing accuracy and safety for diagnosis and treatment of the internal organs of a human body.

An exemplary embodiment of the present invention provides a capsule endoscopy system that includes: a first capsule device that is inserted inside a human body and acquires image data of the inside of the human body that is picked up by a camera; a second capsule device that is inserted inside the human body and performs treatment for the internal organs of the human body; and a control device that is positioned outside the human body, analyzes the image data received from the first capsule device, and transmits a control signal requesting the treatment to the second capsule device on the basis of the analysis results.

Further, another embodiment of the present invention provides a medical system that includes: a first implantable medical device that collects first data for diagnosis in a human body; a second implantable medical device that collects second data for diagnosis in the human body; and a control device that is positioned outside the human body and transmits a control signal requesting second data collection to the second implantable medical device on the basis of analysis results of the first data received from the first implantable medical device.

Further, yet another embodiment of the present invention provides an operation method of a medical system, which includes a plurality of implantable medical devices positioned in a human body and a control device positioned outside the human body, including: forming a network of the plurality of implantable medical devices and the control device using wireless communication; collecting first data of internal organs of a human body by using a first implantable medical device of the plurality of implantable medical devices and then transmitting the first data to the control device; transmitting a first control signal requesting treatment to a second implantable medical device of the plurality of implantable medical devices using wireless communication on the basis of analysis result of the first data by using the control device; and performing treatment for the internal organs of the human body on the basis of the received control signal by using the second implantable medical device.

### BRIEF DESCRIPTION OF THE DRAWINGS

FIG. 1 is a configuration diagram of a medical system according to an exemplary embodiment of the present invention.

FIG. 2 is a view showing a capsule endoscope that is an example of a medical system according to an exemplary embodiment of the present invention.

FIG. 3 is a flowchart illustrating an operation method of a medical system according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

Throughout the specification, unless explicitly described to the contrary, the word “comprise” and variations such as “comprises” or “comprising” will be understood to imply the inclusion of stated elements but not the exclusion of any other elements.

A medical system and an operation method of the medical system according to an exemplary embodiment of the present invention will be described in detail with reference to the accompanying drawings.

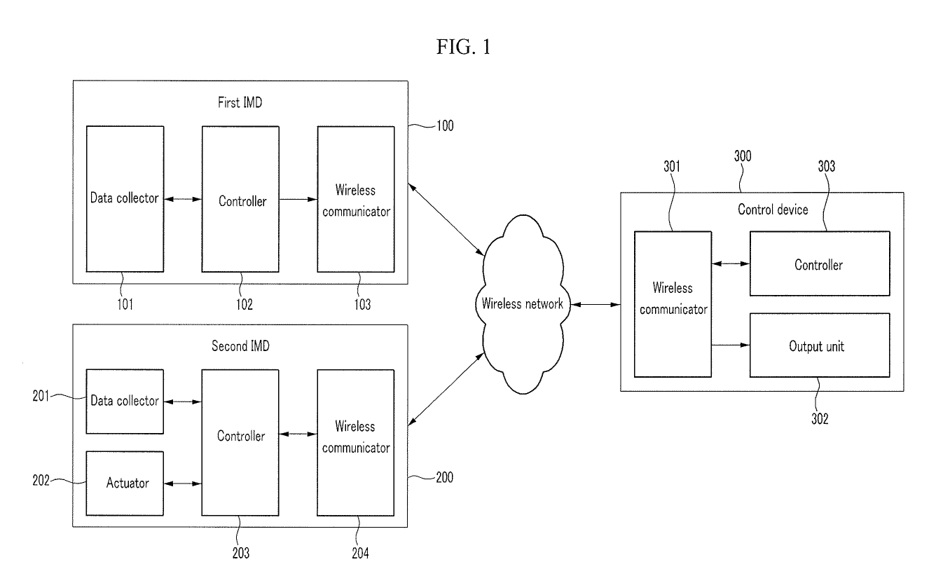

FIG. 1 is a configuration diagram illustrating a medical system according to an exemplary embodiment of the present invention.

Referring to FIG. 1, a medical system according to an exemplary embodiment of the present invention includes a first IMD 100, a second IMD 200, and a control device 300.

The first IMD 100 includes a data collector 101, a controller 102, and a wireless communicator 103, and functions as an investigative device that collects data that is used for medical diagnosis of the internal organs of a human body.

The data collector 101 includes a sensor, a camera, etc., and collects data needed for medical diagnosis of the internal organs of a human body by control of the controller 102.

The controller 102 controls the data collector 101 to collect data needed for diagnosis from the inside of a human body and transmits the data collected by the data collector 101 to the control device 300 outside the human body, using the wireless communicator 103.

The wireless communicator 103 is connected to a wireless network and transmits/receives data to/from the control device 300 outside the human body. In this configuration, the wireless communicator 103 includes one of a low-speed communication module having a data rate of less than hundreds of Kbps (Kilobits per second) (not shown), and a high-speed communication module having a data rate of more than several Mbps (Megabits per second) (not shown), depending on the types of data transmitted/received to/from the control device 300, or may include both the low-speed communication module and the high-speed communication module. For example, in a capsule endoscopy system, the first IMD 100 needs to transmit an image picked up by the camera at a high speed, such that it includes the high-speed communication module. On the contrary, when the first IMD 100 is a glucose sensor, it is possible to transmit sensed data at a low speed, such that it includes the low-speed communication module.

The second IMD 200 includes a data collector 201, an actuator 202, a controller 203, and a wireless communicator 204, and additionally collects data on the inside of a human body in response to a control signal received from the control device 300 through the wireless network, or functions as an identifier device that identifies data previously collected and performs treatment. Meanwhile, although it is exemplified that the second IMD 200 includes both the data collector 201 and the actuator 202 in an exemplary embodiment of the present invention, the second IMD 200 may be configured to include only one of the two constituent elements in the present invention.

The data collector 201 includes a sensor, a camera, etc., and collects data needed for medical diagnosis from the inside of a human body by control of the controller 203.

The actuator 202 performs treatment inside the human body, such as drug delivery, by control of the controller 203.

The controller 203 analyzes a control signal received from the control device 300 through the wireless communicator 204, and controls the data collector 201 to additionally collect data needed for diagnosis from the inside of a human body when additional data collection is needed and then transmits the additionally collected data to the control device 300 outside the human body through the wireless communicator 204. Further, the controller 203 analyzes the control signal received from the control device 300, and controls the actuator 202 to perform treatment inside the human body when treatment is needed.

The wireless communicator 204 is connected to the wireless network, and transmits/receives data to/from the control device 300 outside the human body. In this configuration, the wireless communicator 204, in the same manner as the above wireless communicator 103 of the first IMD, includes one of a low-speed communication module (not shown) and a high-speed communication module (not shown), depending on the types of data transmitted/received to/from the control device 300, or may include both the low-speed communication module and the high-speed communication module.

For example, in a capsule endoscopy system, the second IMD 200 needs to transmit an image picked up by the camera at a high speed or receive a control signal from the control device 300. Therefore, the wireless communicator 204 is configured to include both the high-speed communication module for transmitting image data at a high speed and the low-speed communication module for transmitting/receiving a control signal.

The control device 300 is positioned outside the human body and includes a wireless communicator 301, an output unit 302, and a controller 303.

The wireless communicator 301 is connected to the wireless network and transmits/receives data to/from the first IMD 100 and the second IMD 200 using wireless communication. In this configuration, the wireless communicator 301 includes a plurality of communication modules (not shown), and forms independent communication channels for the IMDs connected to the control device 300 through the plurality of communication modules. That is, communication modules for communication with the first IMD 100 and the second IMD 200 are independently formed. The communication modules can operate as a low-speed communication module or a high-speed communication module in accordance with the characteristics of corresponding IMDs.

The output unit 302 outputs and provides data received from the first IMD 100 through the wireless communicator 301 or additional data received from the second IMD 200 to a manager.

The controller 303 transmits a control signal requesting additional data collection or data identification to the second IMD 200 through the wireless communicator 301 when it is needed to additionally collect data or identify the previously collected data. Further, when treatment inside the human body is needed, the controller 303 transmits a control signal requesting treatment to the second IMD 200 through the wireless communicator 301.

Meanwhile, in an exemplary embodiment of the present invention, the controller 203 can perform a process of determining whether additional data collection is needed, whether identification is needed for the previously collected data, or whether treatment is needed by analyzing the data collected by the first IMD 100, and the manager can identify the collected data through the output unit 302 and determine the process by inputting a control command.

As described above, in an exemplary embodiment of the present invention, since the plurality of IMDs 100 and 200 cooperate with each other by wireless communication, it is possible to improve accuracy and safety in diagnosis and treatment by additionally collecting data, identifying previously collected data, or performing treatment.

Meanwhile, although it is exemplified that two IMDs perform examination, identification, and treatment while cooperating with each other in an exemplary embodiment of the present invention, the present invention can be applied when two or more IMDs perform examination, identification, and treatment while cooperating with each other. For example, when investigation, identification, and treatment are performed by three IMDs, the first IMD can collect data inside a human body, the second IMD can additionally collect data other than the data collected by the first IMD, and the third IMD can perform treatment.

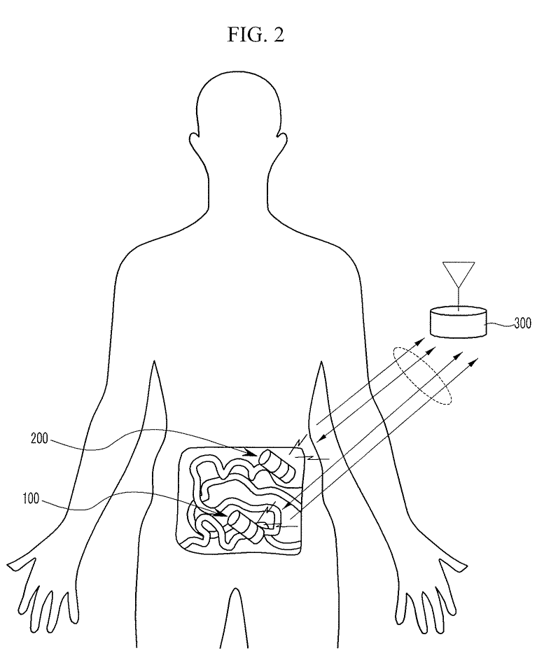

FIG. 2 is a view showing an example of a medical system according to an exemplary embodiment of the present invention, in which a capsule endoscopy system is exemplified.

Referring to FIG. 2, the first IMD 100, which is a capsule device, is inserted inside a human body and transmits data of images of the small intestine, etc., that is picked up by the camera to the control device 300 through the wireless network.

Further, the control device 300 analyzes image data collected by the first IMD 100, and transmits a control signal requesting additional data collection to the second IMD 200 that is a capsule device additionally inserted inside the human body, when image skip occurs while picking up images. Further, the second IMD 200 additionally collects image data obtained by picking up the inside of the human body after analyzing the control signal, and transmits the additionally collected image data to the control device 300.

Further, the control device 300 transmits a control signal requesting treatment, such as drug delivery, to the second IMD 200, when there is an internal organ that needs treatment in the human body as a result of the analysis of the image data collected by the first IMD 100. Thereafter, the second IMD 200 analyzes the control signal and performs treatment, such as drug delivery.

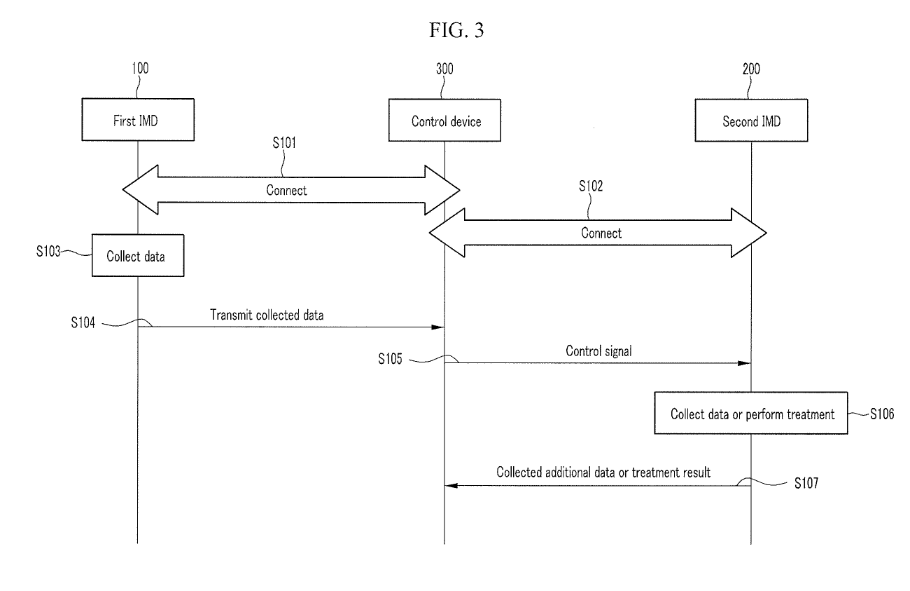

FIG. 3 is a flowchart illustrating an operation method of a medical system according to an exemplary embodiment of the present invention.

Referring to FIG. 3, first, the control device 300 forms a network by performing association using wireless communication with the first IMD 100 and the second IMD 200 inside the human body through the wireless network (S101, S102).

Thereafter, while being normally connected with the control device 300, the first IMD 100 collects data from the internal organs of a human body (S103) and transmits the collected data to the control device 300 using wireless communication (S104).

Thereafter, when it is determined that additional data collection is needed or treatment is needed for an internal organ of a human body as a result of analysis of the data collected by the first IMD 100, the control device 300 transmits a control signal requesting additional data collection or treatment to the second IMD 200 using wireless communication (S105).

The second IMD 200 that has received the control signal analyzes the received control signal, and then additionally collects data or performs treatment for the internal organs of the human body (S106). Further, the second IMD 200 transmits the additionally collected data or the result of treatment to the control device 300 using wireless communication (S107).

Further, the second IMD 200 collects data of the internal organs of the human body and transmits the data to the control device 300 when the control signal is a signal requesting additional data collection, or performs treatment for the corresponding internal organs of the human body and transmits the result of the treatment to the control device 300 when the control signal is a signal requesting treatment.

On the other hand, although it is exemplified that the second IMD 200 collects and transmits data only by the request of the control device 300 in an exemplary embodiment of the present invention, the present invention can be designed to collect data without request of the control device 300, transmit the collected data to the control device 300, and perform treatment only by request of the control device 300. In this case, the control device 300 can use all the data collected by the first IMD 100 and the second IMD 200 for medical diagnosis and request treatment to the second IMD 200 in accordance with the diagnosis result.

According to the embodiments of the present invention, two or more IMD devices form a network together with a control device outside a human body and perform diagnosis and treatment while cooperating with each other, such that it is possible to improve accuracy and safety in the diagnosis.

The exemplary embodiments of the present invention are implemented not only by the method and apparatus, but also by programs that achieve functions corresponding to the configuration of the exemplary embodiments of the present invention or recording mediums including the programs. Further, this can be easily implemented from the description of the exemplary embodiments by those skilled in the art.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A capsule endoscopy system comprising:
a first capsule device that is inserted inside a human body and acquires image data of the inside of the human body which is picked up by a camera;
a second capsule device that is inserted inside the human body and performs treatment for internal organs of the human body; and
a control device that is positioned outside the human body, analyzes the image data received from the first capsule device, and transmits a control signal requesting treatment to the second capsule device on the basis of the analysis result.

2. The capsule endoscopy system of claim 1, wherein the second capsule device has a function of additionally acquiring image data picking up the inside of the human body.

3. The capsule endoscopy system of claim 2, wherein the control device transmits a control signal requesting additional image data collection to the second capsule device, when determining that additional image data collection is needed on the basis of the analysis result.

4. A medical system comprising:
a first implantable medical device that collects first data for diagnosis in a human body;
a second implantable medical device that collects second data for diagnosis in the human body; and
a control device that is positioned outside the human body and transmits a control signal requesting the second data collection to the second implantable medical device, on the basis of analysis result of the first data received from the first implantable medical device.

5. The medical system of claim 4, wherein the first implantable medical device includes:
a data collector that collects the first data using a sensor or a camera;
a wireless communicator that is connected with a wireless network and transmits/receives data to/from the control device; and
a controller that controls the wireless communicator to transmit the first data to the control device.

6. The medical system of claim 5, wherein the wireless communicator includes a low-speed communication module or a high-speed communication module, in accordance with characteristics of the first data.

7. The medical system of claim 4, wherein the second implantable medical device includes:
a data collector that collects the second data using a sensor or a camera;
a wireless communicator that is connected with a wireless network and transmits/receives data to/from the control device; and
a controller that analyzes the control signal received from the control device by wireless communication, and controls the wireless communicator to transmit the second data to the control device.

8. The medical system of claim 7, wherein the second implantable medical device further includes an actuator that performs treatment for the internal organs of a human body, and
the control device transmits a control signal requesting treatment of the internal organs of the human body to the second implantable medical device, on the basis of analysis result of the first data.

9. The medical system of claim 7, wherein the wireless communicator includes a high-speed communication module for transmitting the second data and a low-speed communication module for receiving the control signal.

10. An operation method of a medical system that includes a plurality of implantable medical devices positioned in a human body and a control device positioned outside the human body, the operation method comprising:
forming a network of the plurality of implantable medical devices and the control device using wireless communication;
collecting first data of the internal organs of a human body by using a first implantable medical device of the plurality of implantable medical device, and then transmitting the first data to the control device;
transmitting a first control signal requesting treatment to a second implantable medical device of the plurality of implantable medical devices, using wireless communication, on the basis of analysis results of the first data, by using the control device; and
performing treatment for the internal organs of the human body on the basis of the first control signal, by using the second implantable medical device.

11. The operation method of a medical system of claim 10, further comprising:
transmitting a second control signal requesting additional data collection to the second implantable medical device, using wireless communication, on the basis of analysis results of the first data, by using the control device; and
collecting second data of the internal organs of the human body on the basis of the second control signal and then transmitting the second data to the control device, by using the second implantable medical device.

12. The operation method of a medical system of claim 10, further comprising
collecting second data of the internal organs of the human body and then transmitting the second data to the control device, by using the second implantable medical device,
wherein the transmitting to the second implantable medical device is transmitting the first control signal to the second implantable medical device on the basis of analysis results of the first data and the second data.

13. The operation method of a medical system of claim 10, further comprising
collecting second data of the internal organs of the human body by using a third implantable medical device of the plurality of implantable medical devices and then transmitting the second data to the control device,
wherein the transmitting to the second implantable medical device is transmitting the first control signal to the second implantable medical device on the basis of analysis results of the first data and the second data.

14. The operation method of a medical system of claim 10, wherein the medical system is a capsule endoscopy system, and the first implantable medical device and the second implantable medical device are capsule devices.

15. The operation method of a medical system of claim 14, wherein the first data is image data picking up the internal organs of the human body.
